(19) United States
(12) Patent Application Publication
(10) Pub. No.: US 2004/0023413 A1
(43) Pub. Date: Feb. 5, 2004

(54) Microscale immobilization of molecules using a hydrogel and methods of use thereof

(75) Inventor: Cindra Opalsky, San Diego, CA (US)

Correspondence Address: FOLEY & LARDNER, P.O. BOX 80278, SAN DIEGO, CA, 92138-0278, US

(73) Assignee: Molecular Reflections, Inc.

(21) Appl. No.: 10/306,506

(22) Filed: Nov. 26, 2002

Related U.S. Application Data
(60) Provisional application No. 60/333,591, filed on Nov. 26, 2001.

(51) Int. Cl. C12Q 1/68; G01N 33/543

(52) U.S. Cl. 436/518

(57) ABSTRACT

The present invention provides methods for the immobilization of molecules to a surface using a hydrogel. In particular, the invention relates to the formation and use of a hydrogel in a microscale environment, such as that used for the screening of molecules with microfabricated mechanical resonant sensors. In a preferred embodiment, the molecule is coupled to polyethylene glycol, or a block co-polymer and/or derivative thereof, and immobilized to a planar oxide surface that has been silanized. The immobilized molecule can then be used in a microscale screening or binding assay in an optimal hydrogel environment.

### RELATED APPLICATIONS

This application claims the benefit of U.S. Provisional Application Serial No. 60/333,591, which was filed on Nov. 26, 2001, the contents of which are incorporated by reference herein in their entirety, including all tables, figures, and claims.

### FIELD OF THE INVENTION

The present invention relates to the immobilization of molecules at a surface. More specifically, the invention relates to the immobilization of molecules in a biologically relevant environment, such as a hydrogel, using very small volumes.

### BACKGROUND OF THE INVENTION

The following description is provided to assist the understanding of the reader. None of the information provided or references cited is admitted to be prior art to the present invention.

Solid surfaces are frequently used for the immobilization of a variety of molecules, and molecules immobilized on such surfaces are routinely used for biological research and clinical analyses of specimens. In particular, one member of a binding pair can be immobilized and used to screen samples to analyze the binding properties of cognate binding partners. Examples of typical binding partners include, but are not limited to, antibody/antigen, ligand/ receptor, enzyme/substrate, nucleic acid/nucleic acid. For example, immobilized specific antigens can be used to detect the presence of an immunological response in a biological specimen; and immobilized antibodies can be used to detect and quantify levels of target antigens in samples. As another example, immobilized chemical compounds can be used in screening assays to identify new ligands for known receptor molecules. As a further example, immobilized nucleic acids can be used in hybridization assays to detect and quantify target nucleic sequences in a sample. See, e.g., U.S. Pat. Nos. 4,407,943; 4,895,809; 5,122,452; 5,510,270; 5,766,908; 5,866,387; 6,033,784; each of which is hereby incorporated by reference, including all tables, figures, and claims.

Numerous methods have been described for immobilizing a molecule on a surface. For example, a physical interaction that provides a direct contact of the molecule of interest with the surface, for example, adsorption, can be used. Additionally, a chemical interaction that results in ionic or covalent cross-linking of the molecule to the surface can also be used. For example, U.S. Pat. No. 4,284,553, which is hereby incorporated by reference, including all tables, figures and claims, discloses method for the covalent immobilization of protein molecules to oxide surfaces via thioester-containing coupling chains.

Alternatively, the molecule of interest may be indirectly immobilized on the solid surface. See, e.g., U.S. Pat. Nos. 6,171,610; 6,156,572; 6,048,548; 6,039,977; 5,902,603; 4,452,892, each of which is hereby incorporated by reference, including all tables, figures and claims; which describe methods of indirect immobilization, for example, by “enmeshing” or physically embedding a hydrogel comprising the molecule into a support surface, such as mesh cloth, or porous or roughened surfaces.

Nakayama and Matsuda describe the use of hydrogels to affix molecules to the surface of fabricated devices for use with artificial organs (Nakayama and Matsuda, ASAIO J. 39:M754-757, 1993). Hydrogel coated on such devices may, for example, contain therapeutic molecules such as heparin, which is then released into the body from the hydrogel to inhibit platelet adhesion. U.S. Pat. No. 5,028,339, which is hereby incorporated by reference, including all tables, figures and claims, describes a method for holding a reactant in a polymer matrix, by polymerizing the matrix around the reactant without chemically attaching to or embedding the reactant. The polymer matrix can be sorbed or coated on a substrate, cast into shapes or otherwise made available for reaction. U.S. Pat. No. 4,518,693, which is hereby incorporated by reference, including all tables, figures and claims, describes the immobilization of biocatalysts such as microbial cells by forming spherical gel beads containing the cells from a hydrogel such as agar or carrageenan.

### SUMMARY OF THE INVENTION

The present invention provides novel methods and compositions for the immobilization of molecules at a surface. Preferably, the molecule is immobilized in a biologically relevant aqueous or semi-aqueous environment, such as a water-swellable hydrogel, to allow the molecule to freely interact with other molecules, and to mimic its native environment. Once immobilized to a surface, the molecule can be used as an immobilized binding partner. Such an immobilized molecule can, for example, be contacted with a test sample to determine if the sample contains a binding partner for the immobilized molecule. The methods and compositions used herein can be used in a variety of ways, for example, to screen samples to identify an unknown binding partner, to test samples for the presence and/or quantity of a molecule known to be a binding partner, or to analyze the binding interaction between binding partners. The invention further contemplates the use of these methods and compositions in a microscale environment, using volumes below the microliter range, and preferably below the nanoliter range. In a preferred embodiment, the methods and compositions of the invention can be used in microfabricated sensor devices to detect binding events between molecules by monitoring changes in mass which result in changes in the physical resonance of such a sensor.

In various embodiments, chemical immobilization can be used to couple a hydrogel to one or more molecules and/or to immobilize the hydrogel to a surface. This may allow one to physically manipulate the surface in assay procedures without the problems related to leaching of the molecule from the surface, and/or dislodging of the hydrogel from the surface for example. In addition, this method of hydrogel immobilization of a molecule can be advantageous to screening and/or diagnostic methods, and in particular high-throughput assays, where the goal is to retain the molecule within the hydrogel at a solid surface for detection or binding. The use of the hydrogel environment allows the molecule of interest to freely move and rotate and prevents excessive undesired interaction with the surface and/or undesired lateral interactions between neighboring molecules. As a result, the molecule of interest is optimally accessible for further interaction, e.g. with a binding partner.

In a first aspect, the invention relates to methods for the microscale immobilization of molecules at a discrete location on a surface. The hydrogel is formed from a plurality of linkers, wherein one or more linker(s) binds to the molecule of interest, and one or more linker(s) bind to the surface. Preferably, the immobilized hydrogel comprising the molecule of interest has a volume of 1 microliter or less.

The term “microscale” as used herein refers, to the use of the invention hydrogel in an environment where solutions are typically manipulated in very small volumes, for example, less than a microliter (μl). The term is not meant to limit the methods or compositions of the present invention to a particular range, but rather to signify experimentation on a very small physical scale. Considering this volume of solution, it is preferable that the hydrogel of the present invention is immobilized rapidly to minimize the chance of evaporation of solutions. In preferred embodiments, the microscale hydrogel occupies a volume of from 0.1 picoliters to 1 microliter, more preferably from 1 picoliter to 100 nanoliters, more preferably from 10 picoliters to 1 nanoliter.

The term “immobilization” as used in a first context refers to the coupling of a hydrogel comprising a molecule to a surface. In as second context, immobilization of a molecule refers to the indirect attachment of a molecule to a surface via a hydrogel. Numerous methods are known to the skilled artisan to achieve such coupling. For example, this immobilization can be accomplished through chemical or photo-activated means. In a preferred embodiment the coupling is covalent. In a more preferred embodiment, the hydrogel can be attached to the surface via hydroxyl groups at the planar surface. The immobilization may be reversible, or preferably permanent.

The term “hydrogel” as used herein refers to a gelatinous colloid, or aggregate of molecules in a finely dispersed semi-liquid state, where the molecules are in the external or dispersion phase and water is in the internal or dispersed phase. Preferred hydrogels include compositions that allow molecules in environment surrounding the hydrogel to readily diffuse or permeate the entire hydrogel. See, e.g., U.S. Pat. Nos. 6,268,161; 5,985,320; 5,798,113; 5,644,049; 5,275,838; 4,959,148; 4,699,946; 5,972,199; 5,766,908; 5,057,421; 4,956,289; 4,732,851; 5,733,563; each of which is hereby incorporated by reference, including all tables, figures and claims. Presently preferred hydrogels are made using polyethylene glycol, polypropylene glycol or polylysine, or a derivative (such as a branched or star molecule) or block co-polymer thereof.

The term “linker” as used herein refers to a molecule containing a functional group on at least one end through which the molecule can be coupled and/or the linker can be coupled to the surface. The linker can be a simple linear molecule with two ends, or a composite or three-dimensional molecule, such as a branched or star molecule with multiple ends, which can potentially contain the functional group. In various embodiments, the functional group may be photo-activatable or chemically activatable and/or comprise an acrylic (e.g., comprising —C₂H₃) or allylic group (e.g., comprising —C₃H₅). Presently preferred linkers may contain an acrylic or allylic group that is photo-activatable on one end, a hydrophilic spacer, and a functional coupling group for attachment to the molecule of interest. In a more preferred embodiment, the linker comprises polyethylene glycol (PEG), a branched PEG, star PEG, or a block co-polymer or derivative thereof. In alternative embodiments the linker comprises polypropylene glycol (PPG), a branched PPG, star PPG, or a block co-polymer or derivative thereof, polylysine or a block co-polymer or derivative thereof, agarose or acrylamide. Combinations or mixes of various forms of such linkers may also be used.

The phrase “linkers bind said molecule” as used herein refers to the attachment of linker to the molecule by any means that eventually couples the two. For example, the coupling may be covalent, chemical crosslinking or photo-crosslinking. Cross-linking agents are well known to the skilled artisan. See, e.g., U.S. Pat. Nos. 6,011,077; 5,853,744; 5,660,692; 5,431,790; 6,277,570; 5,185,433; 5,583,211; each of which is hereby incorporated by reference, including all tables, figures and claims. In addition, the coupling may be direct, for example a PEG linker to the molecule of interest, or indirect, for example, a PEG linker to a biotin moiety to a streptavidin-derivatized molecule of interest. Multiple levels of indirect coupling may also be used, for example, a PEG linker to a biotin moiety, attached to a streptavidin, attached to a biotinylated molecule of interest.

The hydrogel of the present invention comprising the molecule can be immobilized to the surface of a substrate. The surface is preferably flat or planar, although alternative surfaces may be angled, sloped or curved. Any shape of surface may be used, including but not limited to, square, rectangular, round oval or polygonal shapes. In a preferred embodiment, the surface area is less than 1 cm², more preferably less than 0.5 cm², more preferably less than 0.1 cm². Appropriate substrate compositions include, but are not limited to, compositions comprising single crystal silicon, polysilicon, silicon nitride, silicon dioxide, phosphosilicate glass, borophosphosilicate glass, aluminum nitride, zinc oxide, polyvinylidene fluoride, lead zirconate, metal (such as titanium, metal alloy, gold, platinum, tungsten, aluminum), aluminum oxide, tin oxide, tantalum oxide, and the like. Combinations of these materials also can be used. The substrate surface may further comprise an intermediate layer to facilitate attachment of the hydrogel, for example, by providing additional reactive groups. In preferred embodiments, the hydrogel is not capable of penetrating the surface. In one embodiment the surface comprises a microfabricated resonant sensor.

In a preferred embodiment, the surface to which the immobilized molecule and hydrogel is attached is a surface of a microfabricated mechanical resonant sensor. An example of such a device is described in the Patent Application filed with the USPTO on Sep. 20, 2001, entitled “Microfabricated Ultrasound Array for Use as Resonant Sensors” (U.S. application Ser. No. 09/957,875), the contents of which are hereby incorporated by reference in their entirety, including all tables, figures, references and claims. Microfabricated resonant sensors can be used individually or as an interconnected yet electrically isolated grouping in microarrays. Electromechanical sensors can be used to monitor a change in surface properties of a sensor membrane. The change in surface properties results from a binding event that changes the physical characteristics of the membrane surface, such as surface mass, viscous coupling, membrane stiffness, and the like. These sensors can also be used to determine a change in force on the surface of a sensor membrane, such as results from a binding event or application of pressure. A sensor can be part of an array of sensors that can be fabricated to high density. These sensors have many applications including, for example, to determine the presence or amount of an analyte in a sample from a clinical, research or natural environment. In this case, a binding partner of the analyte can be immobilized to the resonant sensor membrane surface and the binding of analyte to the binding partner on the membrane can be identified through a shift in the resonant characteristics of the sensor membrane.

The term “sensor” as used herein refers to an apparatus or device that can respond to an external stimulus such as, a change in mass on a surface, pressure, force, or a particular motion, where the apparatus can transmit a resulting signal to be measured and/or detected.

As used herein “microfabricated” refers to the procedures and/or methods, such as bulk and surface micromachining, used to etch, deposit, pattern, dope, form and/or fabricate structures using substrates such as silicon and the like. Microfabrication procedures are known in the art and have been used to prepare microsystems such as computer processor chips, acoustic sensors, micro-circuits and other devices requiring micron and nanomolecular scale portions used in fields such as microengineering.

The term “discrete location” as used herein refers to a physical spot on the surface to which a volume of up to 1 microliter can be applied as an aqueous drop. The drop applied is preferably a volume of from 0.1 picoliters to 1 microliter, more preferably, 1 picoliter to 100 nanoliters, more preferably 10 picoliter to 1 nanoliter. In further embodiments, a plurality of drops may be applied to one discrete location; and/or a plurality of drops may be applied to a plurality of discrete locations.

A molecule as used herein includes any molecule that one intends to detect or further analyze. Although a molecule can be obtained from any source, in certain embodiments, the molecule is a biological molecule such as a polypeptide, peptide, chemical compound, antibody, polynucleotide or carbohydrate. Molecules of particular interest may include, but are not limited to, antibodies, antigens, nucleic acids, lectins, sugars, oligosaccharides, glycoproteins, receptors, growth factors, cytokines, molecules such as drug candidates (from, for example, a random peptide library, a natural products library, a legacy library, a combinatorial library, an oligosaccharide library and a phage display library), metabolites, drugs of abuse and their metabolic by-products, enzyme substrates, enzyme inhibitors, enzyme co-factors such as vitamins, lipids, steroids, metals, oxygen and other gases found in physiologic fluids, cells, cellular constituents, cell membranes and associated structures, cell adhesion molecules, natural products found in plant and animal sources, tumor markers (i.e., molecules associated with tumors), other partially or completely synthetic products, and the like.

In order to incorporate a molecule within the hydrogel, the molecule is coupled to one or more linkers. As used herein, the term “coupling” or “coupled” refers to a linkage (preferably covalent) of the molecule to the linker and/or a linkage of the linker to the surface. In preferred embodiments, the molecule is covalently coupled to one or more linkers to avoid leaching of the molecule from the resulting hydrogel in further uses. In various embodiments the molecule may be bound to the linker molecule using photo-crosslinking or chemical crosslinking, and may be direct or indirect as described above. In addition, the molecule may be derivatized to incorporate a reactive group, for example a photo-activatable functional group, to achieve coupling to the linker. Preferably, the molecule may be coupled to the linker prior to polymerization of the linkers to form the hydrogel. Alternatively, the molecule may be coupled a hydrogel that has been formed by polymerizing a plurality of linkers. In preferred embodiments, one or more linkers are covalently coupled to the surface. In various embodiments one or more linkers may be bound the surface using photo-crosslinking or chemical crosslinking. One or more linkers may be contacted with the surface to form the immobilized hydrogel and/or one or more linkers can be polymerized to form a hydrogel to be contacted with the surface to form the immobilized hydrogel.

In certain embodiments the molecule is a polypeptide or peptide. As used herein, a “polypeptide or peptide” is a chain of covalently linked amino acids, regardless of length or post-translational modification (e.g., glycosylation, phosphorylation, or attachment of lipid). A polypeptide, protein or peptide is essentially a polymer in which the monomers are amino acid residues that are joined together through amide bonds. Proteins may include non-covalent linkages of polypeptides (e.g., disulfide cross-linkages). The polypeptide or peptide of the invention may be naturally occurring, as well as those that are recombinantly or synthetically synthesized. Polypeptide or peptide fragments are also encompassed by the invention. Polypeptides of particular interest may include, but are not limited to, receptors, growth factors, cytokines, enzymes and disease markers or probes. In addition, molecules of interest, which may or may not be polypeptides, include enzyme substrates, enzyme inhibitors and enzyme cofactors.

In certain embodiments the molecule is a chemical compound. As used herein, a “chemical compound” is any substance formed by the covalent or electrostatic union of two or more elements. A non-limiting compilation of chemical compounds is provided in The Merck Index. This term is intended to include potential and existing drugs and pharmaceuticals, organic and inorganic chemicals, laboratory reagents, and naturally occurring or synthetic agents. A library of chemical compounds can be prepared or commercially obtained, for example, as a combinatorial library. Chemical compounds of particular interest may include, but are not limited to pharmaceutical compositions, metabolites, drugs of abuse and metabolic by-products thereof. Preferably, chemical compounds are small molecules, more preferably small molecules with a molecular weight less than 1000.

In certain embodiments the molecule is an antibody. As used herein, an “antibody” is an immune or protective protein, such as is typically evoked in an animal in response to an immunogen or antigen. This term is intended to include all forms of an antibody, including all natural and unnatural antibody forms, and any and all fragments and derivatives thereof. This includes the typical antibody that consists of four subunits including two heavy chains and two light chains, domain-deleted antibodies, Fab fragments, Fab′2 fragments, Fv fragments, single chain Fv antibodies (see, e.g., U.S. Pat. No. 5,840,300, which is hereby incorporated by reference, including all tables, figures and claims), antigen binding sites, and the like. An antibody also includes the heavy chain alone or the light chain alone. Methods to produce polyclonal antibodies and monoclonal antibodies are well known in the art (see, e.g., Harlow and Lane, “Antibodies, a laboratory manual.” Cold Spring Harbor Laboratory, 1988). Non-naturally occurring antibodies can be constructed using solid phase peptide synthesis, can be produced recombinantly or can be obtained, for example, by screening combinatorial libraries comprising variable heavy chains and variable light chains as is known in the art (see, e.g., U.S. Pat. No. 5,565,332, which is hereby incorporated by reference, including all tables, figures and claims).

In certain embodiments the molecule is a polynucleotide. As used herein, a “polynucleotide” is a linear polymer containing any number of component nucleotides, typically linked from one ribose (or deoxyribose) moiety to another via phosphoric residues. A polynucleotide may be natural or synthetic, and may be single-stranded or double-stranded. This term is intended to include deoxyribonucleotides (DNA), and ribonucleotides (RNA) in which uracil (U) is present in place of thyrnidine (T), or alternative forms of either nucleotide, such as genomic DNA (gDNA), complementary DNA (cDNA), messenger RNA (mRNA) and transfer RNA (tRNA). In preferred embodiments, the polynucleotide is an oligonucleotide less than 100 nucleotides in length, more preferably, less than 50 nucleotides in length. A polynucleotide also includes non-traditional nucleic acids with altered backbone structures, such as peptide nucleic acid (PNA) and locked nucleic acid (LNA) oligomers (see, e.g., Elayadi and Corey, Curr. Opin. Invest. Drugs 2:558-561, 2001).

In certain embodiments the molecule is a carbohydrate. As used herein, a “carbohydrate” is any form of saccharide. Examples of carbohydrates include, but are not limited to, simple sugars or oligosaccharides (such as monosaccharides, disaccharides, etc. which have typical molecular weights less than 1000) as well as macromolecular (polymeric or polysaccharides) substances such as starch, glycogen, and cellulose polysaccharides (which may have molecular weights on the order of 10⁵-10⁶).

In a second aspect, the invention relates to methods and compositions for screening immobilized molecules using the hydrogel. Such methods can comprise contacting the hydrogel containing the molecule, which is immobilized to a surface, with a sample suspected of containing or known to contain a binding partner for the molecule within the hydrogel and detecting a binding event between the molecule and its binding partner.

The term “binding event” refers to an interaction or association between a minimum of two molecular structures, such as a first molecule and a second molecule that is a binding partner for the first molecule. The interaction may occur when the two molecular structures are in direct or indirect physical contact. Examples of binding events of interest in the present context include, but are not limited to, ligand/ receptor, antigen/ antibody, enzyme/substrate, DNA/DNA, DNA/RNA, RNA/RNA, nucleic acid mismatches, complementary nucleic acids, nucleic acid/proteins, and the like. The binding affinity of such interactions may be measured to determine a dissociation constant (Kd). Typical Kd values for such interactions are 10⁻⁵M, 10⁻⁶M, 10⁻⁷M, 10⁻⁸M, 10⁻⁹M, 10⁻¹⁰M, 10⁻¹¹M, and 10⁻¹²M. A binding event may be detected by various means known to one skilled in the art, for example, by detecting a chemically-generated color change, fluorescence or luminescence.

The term “screening” as used herein refers to the determination of any interaction between two binding partners, i.e., between a target and a second molecule which have the ability to interact, one of which is incorporated in the hydrogel. In a preferred embodiment the target molecule within the hydrogel is screened against a plurality of unknowns which are potential binding partners. A plurality in this context means more than 1 potential binding partner in the same sample, preferably more than 10, more preferably more than 100, more preferably more than 1000, more preferably more than 10,000.

For example, in one embodiment, one or more molecule(s) incorporated within the immobilized hydrogel constitutes a target. A sample that is suspected of containing, or known to contain a binding partner for this target is then used to contact the hydrogel. This method could be used, for example, to identify and/or characterize a receptor molecule for a molecule ligand. A positive “hit” or match would be detected by a binding event between a test molecule and a target molecule.

A “sample” as used herein refers to essentially any source from which a binding partner can be obtained. A sample can be in a liquid or a gaseous phase, or it may also be in a more solid phase. A sample may be acquired from essentially any organism, including animals and plants, as well as cell cultures, recombinant cells, cell components and can also be acquired from environmental sources. A “biological sample” as used herein refers to a sample obtained from a biological tissue, fluid or specimen and may be obtained from a diseased or healthy organism. Biological samples may include, but are not limited to, sputum, amniotic fluid, blood, blood cells (e.g., white cells), serum, plasma, urine, semen, peritoneal fluid, pleural fluid, saliva cerebrospinal fluid, tissue or fine needle biopsy samples, tissue homogenates and tissue culture media. Samples may also include sections of tissues such as frozen sections taken for histological purposes. Typically, samples are taken from a human. However, samples can be obtained from other mammals also, including by way of example and not limitation, dogs, cats, sheep, cattle, and pigs. The sample may be pretreated as necessary by dilution in an appropriate buffer solution or concentrated, if desired. Any of a number of standard aqueous buffer solutions, employing one of a variety of buffers, such as phosphate, Tris, or the like, preferably at physiological pH can be used.

A sample also may be artificially prepared. For example, a control sample that contains a known amount of an analyte can be prepared for comparative use. In addition, another type of sample may include a source suspected of containing a binding partner for the molecule of interest, such as a preparation of a plurality of proteins or chemical compounds.

The term “environmental sources” includes potentially any place in the natural and/ or man-made environment from which a sample can be taken. Environmental sources include, but are not limited to: water sources such as oceans, lakes, ponds, rivers and streams; earthen sources such as soil, sand, interior or exterior dust; gas sources such as air, such as polluted and/or non-polluted air from our general surroundings or from industrial plants or automotive exhaust and the like.

Biological samples can be derived from patients using well-known techniques such as venipuncture, lumbar puncture, fluid sample such as saliva or urine, or tissue biopsy and the like. Biological samples also include exhaled air samples as taken with a breathalyzer or from a cough or sneeze. A biological sample may be obtained from a cell or blood bank where tissue and/or blood are stored, or from an in vitro source, such as a culture of cells. Techniques for establishing a culture of cells for use as a source for biological materials are well known to those of skill in the art.

As discussed above, the binding event can be between biological or chemical molecules and can be obtained from a variety of samples. In order to achieve a binding interaction, various assays that rely on the binding of one molecule to another can be utilized. Fields such as immunology, pharmacology, biology, medicine, chemistry, molecular biology and other like fields of science have long utilized assays involving molecular and chemical binding events. Such assays including ELISA, DNA hybridization, immunoassay, competitive binding assays, sensitivity assays, affinity and rate binding assays and the like, rely on detection of optically or chemically detectable indicators such as fluorescent, luminescent, or other visible markers which are bound to one member of a binding pair, or direct measurements such as changes in refraction, polarization, ellipticity, etc., of light. Such assays are routine in the art and can be found in various publications, e.g., Current Protocols in Immunology, Eds. Coligan et al.; Current Protocols in Molecular Biology, Eds. Ausubel et al.; and Current Protocols in Pharmacology, Eds. Enna et al.; all published by John Wiley & Sons, Inc., each of which is hereby incorporated by reference, including all tables, figures and claims. Also see, e.g., U.S. Pat. Nos. 6,004,755; 6,215,894; 6,287,783; 5,783,399; 4,629,690; each of which is hereby incorporated by reference, including all tables, figures and claims.

The sensor or sensor arrays can perform these same assays and can optionally use the same marker labeled reagents. In a preferred embodiment where the hydrogel is immobilized to a microfabricated resonant sensor, the result of a binding interaction, i.e. a binding event, is appreciated by detecting a change in the surface supporting the immobilized hydrogel containing the molecule. This change may represent a change in mass at a membrane surface of the sensor or a plurality of sensors in a sensor array. Preferably the change in mass is directly related to a binding event on or near the surface of the membrane.

The change in the sensor membrane that supports the hydrogel containing the immobilized molecule results in a membrane response. As used herein the term “membrane response” relates to the vibration or resonance of the membrane layer that is extended over, or placed on, and roughly covers, in a sealed liquid impermeable, manner a cavity of the invention sensor. Upon the introduction of a current or formation of an electrostatic potential, the membrane of the invention can move, vibrate or oscillate in a manner that can be measured, for example, acoustically, electronically by electromechanical transduction such as by electrostatics/capacitance, piezoresistance or piezoelectricity, or optically by interferometry, such as laser-Doppler vibrometery. The extent of vibration or oscillation of the membrane depends, for example, on the physical properties of the membrane and its relation to another electrode in the cavity or the effect of mass or force on the membrane surface.

While the invention described herein has been discussed in terms of comprising sensors and arrays that can be used with our without detectable labels, it is important to understand that the term “detectable labels” refers to labels as normally used in the art to detect a bound analyte through the use of chemical, radioactive and/or optical means. However, the present invention can use detectable labels that specifically relate to aspects of the present invention. With regard to the present invention, a detectable label can also be a molecule or substance that is attached to a binding partner for an analyte of interest, or a binding partner which binds to an analyte of interest that adds a certain amount of additional mass to make the detection for readily detected. Thus, a detectable label of the invention can be a label that adds a particular amount of additional molecular mass to a bound pair, or deposits, upon enzymatic reaction, a detectable amount of molecular material to the surface of the substrate in response to a probe or a target that is bound thereon, such as to provide an amplification of the original binding interaction.

In a third aspect, the invention relates to methods and compositions for detecting and/or analyzing a binding event between an immobilized molecule and a binding partner therefor. These invention methods comprise contacting the hydrogel immobilized to a surface, with a sample suspected of containing or known to contain a binding partner for the molecule within the hydrogel and detecting a binding event between the molecule and its binding partner. The binding events and samples are the same as described above for the screening methods.

In a preferred embodiment, the method detects and/or analyzes a binding event between an immobilized molecule and a binding partner therefor within a hydrogel that is contained on a microfabricated mechanical resonant sensor. Examples of such analysis include, but are not limited to: the determination of a rate of binding between a binding partner within a sample and the immobilized molecule; and the determination of affinity between a binding partner within a sample and the immobilized molecule. The device and detection methods are the same as described above for the screening methods.

In a fourth aspect, the invention relates to a hydrogel for the microscale incorporation of molecules that can be immobilized at a surface. In preferred embodiments this hydrogel can be applied to the surface with a thickness of less than about 3000 Å, preferably less than about 1500 Å, more preferably less than about 750 Å, more preferably less than about 500 Å. In preferred embodiments the hydrogel occupies a volume of from 0.1 picoliters to 1 microliter, more preferably, 1 picoliter to 100 nanoliters, more preferably 10 picoliter to 1 nanoliter. The hydrogel is formed by the methods described above, and incorporates all the properties and embodiments discussed.

In a fifth aspect, the invention relates to a library of molecules incorporated within a hydrogel, preferably at discrete addressable locations. In a preferred embodiment, the molecules within the hydrogel retain biological activity. As used herein, a “library” refers to a plurality of distinct molecules. Preferably, a library contains more than 10 molecules. More preferably, a library contains more than 100 molecules. Most preferably, a library contains more than 1000, more than 5000, more than 10,000, more than 25,000, more than 50,000, more than 100,000, or more than 1,000,000 distinct molecules. Examples of libraries include, but are not limited to peptide libraries, natural product libraries, legacy libraries, oligosaccharide libraries, phage display libraries, and the like.

Preferably, the molecule retains biological activity upon being incorporated into the hydrogel of the invention. The term “biological activity” refers to the characteristic properties of a molecule that are present prior to attachment to the linker or incorporation into the hydrogel. For example, the molecule within the hydrogel should retain its native binding or enzymatic properties in order to be further characterized or screened. When a molecule has more than one biological activity, it preferably retains one or more, but not necessarily all of such activities. For example, a molecule may retain one activity (such as the ability to bind a partner) and lose another activity (such as enzymatic activity) and still retain biological activity.

In a sixth aspect, the invention relates to an apparatus for the detection and/or analysis of a binding event between a molecule and a binding partner therefor. The invention apparatus comprises the hydrogel and the immobilized molecule as described. In a preferred embodiment, the apparatus is a microfabricated resonant sensor devise or a derivative or improvement thereof.

The following description of the figures and the figures provided herein are meant to assist the understanding of the reader are not intended as limitations on the scope of the invention.

### BRIEF DESCRIPTION OF THE FIGURES

FIG. 1 illustrates an exemplary coupling of a molecule to a linker. R represents the molecule; X and Y represent the functional groups to be used in coupling.

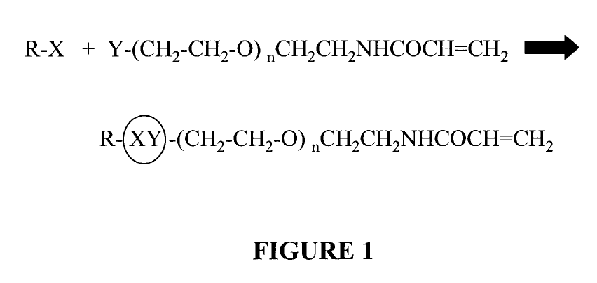

FIG. 2 illustrates possible combinations of functional groups to couple a molecule to a linker. R represents the molecule; X and Y represent the functional groups to be used in coupling.

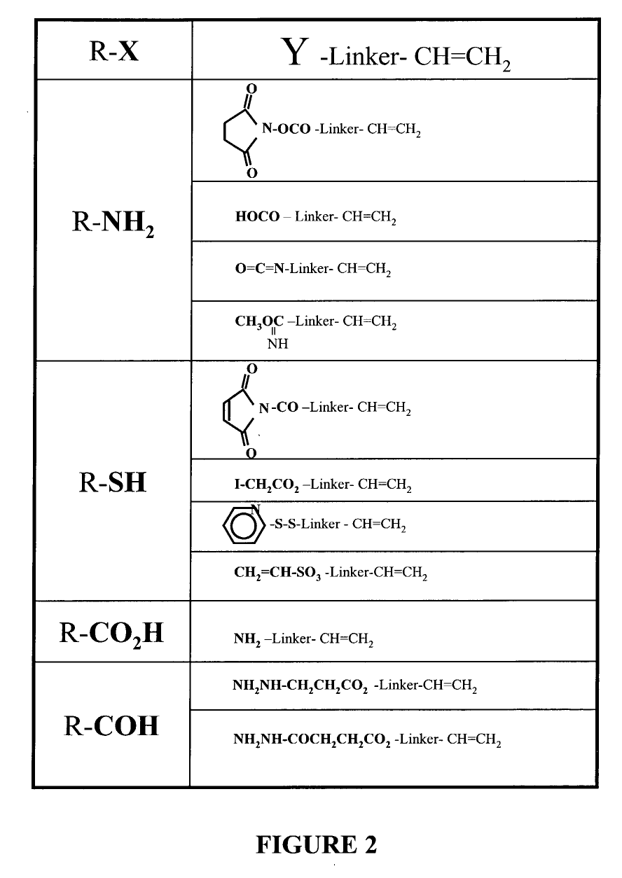

FIGS. 3A to 3C collectively illustrate the use of photo-crosslinking to immobilize a hydrogel comprising a molecule on a planar surface. FIG. 3A shows the deposition of a solution containing a molecule of interest (R) attached to a photopolymerizable linker, a linker with photopolymerizable groups attached at both ends, and a photoinitiator. FIG. 3B shows the application of a radiation source to the droplet to activate the photopolymerizable groups. FIG. 3C shows the resultant immobilized hydrogel comprising covalently linked molecules (R).

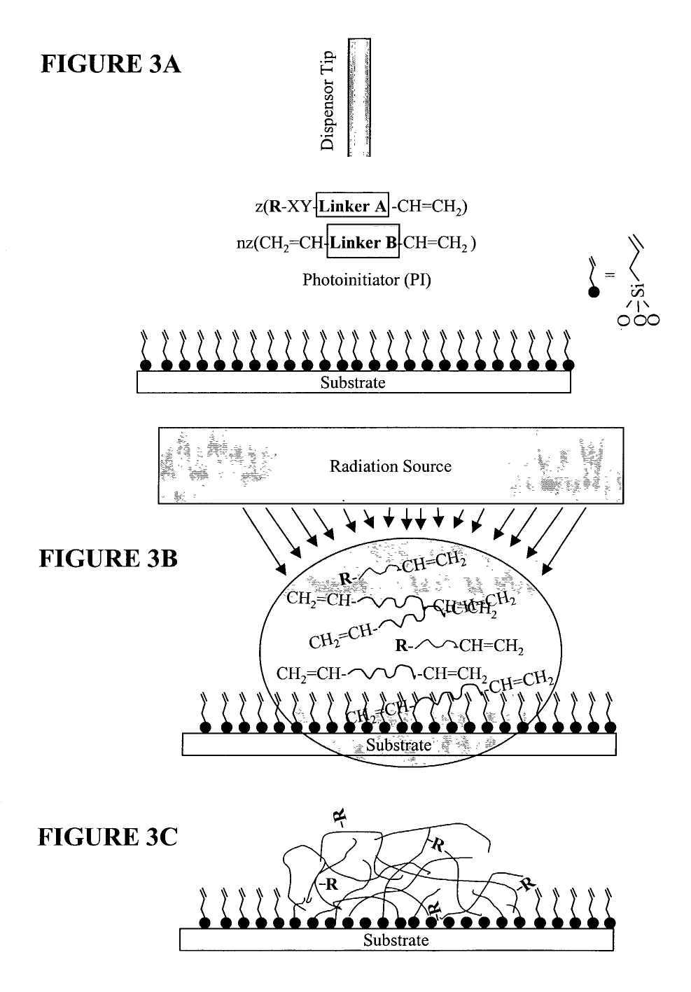

### DETAILED DESCRIPTION OF THE INVENTION

In accordance with the present invention, there are provided methods and compositions for the microscale immobilization of molecules at a surface using a hydrogel and methods of use thereof. In one aspect, the invention provides methods for the microscale immobilization of a molecule at a surface where a hydrogel comprising a molecule is formed from a plurality of linkers. One or more of the linkers bind the molecule, and one or more of the linkers bind the surface. In further various aspects, the invention provides methods of screening and analyzing such immobilized molecules and libraries thereof, and apparatus comprising such immobilized molecules.

Hydrogels

Typical hydrogels are stable (i.e., resistant to dissociation) in water, and most preferably can be water-swellable, i.e., they can absorb water. This water absorption is beneficial because incorporated molecules can be kept in a “water-like” or aqueous environment. This aqueous environment can provide a biologically relevant environment for the molecules within the hydrogel, permitting such molecules to maintain native conformations and interact with external aqueous solutions, for example biological samples, subsequently applied to the hydrogel. The linkers of the present invention form the substance of the hydrogel.

The hydrogel of the present invention is typically formed of linkers comprising polyethoxy groups (i.e. polyethylene glycol groups on “PEG”). Preferred molecules include nonlinear linker molecules such as branched PEG, star PEG and other derivatives of PEG. Alternative embodiments include, but are not limited to, alternative alkylene polyethers, poly-lysine, polypropylene glycol, block co-polymers and/or derivatives thereof, agarose and acrylamide. A block co-polymer can be made by linking individual polyalkeneoxy groups, polypropoxy groups, polybutoxy groups, and the like, using urethane-urea groups. Chains of these molecules (also referred to as linkers) are cross-linked to create the hydrogel. A general discussion of PEG hydrogels can be found in “Poly(ethylene glycol) Gels and Drug Delivery” by N. B. Graham, Chapter 17, p. 263-281 in “Poly(ethylene glycol) chemistry: Biotechnical and Biomedical Applications, Ed. J. M. Harris, 1992. Although the present invention is described in terms of PEG, the skilled artisan will understand that other polyalkeneoxy molecules can be used as linkers, either replacing or together with PEG.

PEG is made from ethylene oxide by anionic polymerization that leaves a hydroxyl on each end of the molecule for polymerization. Among alkylene polyethers, poly(ethylene oxide) units have significant solubility in water at all molecular weights. Thus, PEG is soluble in aqueous media, and in water, PEG chains are not very reactive with other compounds present. Thus, PEG is ideal to minimize non-specific adsorption of the molecule to the surface. In addition, as a hydrogel, the polymerized PEG molecules are stable and resistant to degradation due to contact with further solutions.

In various embodiments, the composition of the hydrogel can be varied by altering the properties of the linker used. For example, the porosity of the hydrogel can be controlled and varied by changing the molecular weight of the linker component to allow maximal accessibility of the particular molecule being surrounded. In addition, variations in molecular weight can be achieved by varying the length of the linker arms. Optimally, the porosity of the hydrogel is such that molecules of interest in a solution can access the molecules within the hydrogel. The porosity of the hydrogel can be analyzed by measuring the diffusion rate of labeled molecules of varied size. Such molecules can incorporate optically active or fluorescent dyes. In order to allow large molecules to diffuse into the hydrogel, the diffusion coefficients of such molecules are optimally within an order of magnitude of solution diffusion coefficients. Changes in porosity are achieved, for example, by varying the molecular weight and/or concentrations of the PEG linkers used.

According to the methods of the present invention, the hydrogel is preferably composed of unit linker structures with molecular weight ranges of 100 to 100,000, preferably 1000 to 50,000, more preferably 5000 to 25,000, more preferably, 10,000 to 20,000. The preferred size of the linker is in the range of about 1-15 PEG molecules, preferably 1-10 PEG molecules, most preferably 2-6 PEG molecules. The water content of such a hydrogel is preferably 30-95%, more preferably 50-90%, most preferably 70-85%. An exemplary PEG concentration is in the range of 5-60% PEG weight/volume, more preferably 10- 50% PEG weight/volume, more preferably 15-40 PEG % weight/volume, most preferably 20-30% PEG % weight/volume. The linkers may be prepared, for example, in a variety of aqueous solvents, ethanol, methanol, dimethylsulfoxide (DMSO), and the like, or combinations thereof.

As discussed below, the component units of the hydrogel can become cross-linked to form the hydrogel by allowing chemical interactions to occur spontaneously on addition of water or a water-based solution, or by photo-polymerization or photo-crosslinking.

Coupling of a Molecule of Interest to a Linker

In the incorporation of the molecule within the hydrogel a long linker arm may be used to provide a physical distance separation of the molecule from the surface. This physical distance can be measured in terms of ethylene oxide units, for example, the number of units in the repeat structure of a linker such as PEG. The molecule can be separated from the surface in the range of 20-1250 ethylene oxide units, more preferably 100-500 ethylene oxide units, most preferably 200-400 ethylene oxide units. This can allow the molecule to rotate and may increase accessibility of the molecule to molecules of interest (e.g., probing agents). By moving the molecule away from the surface, non-specific adsorption of the molecule to the surface may also be minimized. The hydrogel environment can be used to physically separate molecules from one another to reduce or eliminate lateral interaction between neighboring immobilized molecules. Optimally, the local concentration of the molecule within the hydrogel is kept low enough such that the molecules are completely enveloped and neighboring interaction between molecules are eliminated. Preferably, the local concentration of the molecular is less than 1 millimolar, more preferably less than 100 micromolar, most preferably less than 10 micromolar.

Preferably the coupling reaction between the linker and the molecule is carried out in solution prior to the addition of these compounds to the surface. This is desirable because the coupling reactions can be slow, and considering the use of the hydrogel in a microscale environment, evaporation is likely to occur before a coupling between the compounds is achieved. In addition, the separation of the two coupling reactions (i.e. the coupling of the molecule to the linker and the immobilization of the linker comprising the hydrogel to the surface) is preferable to optimize each coupling reaction and minimize undesired interactions between molecules and non-specific adsorption of molecules to the surface.

The concentration of the molecule can be varied within the hydrogel. This is achieved by adding diluent linker molecules to the already coupled molecule-linker combination. Concentrations are controlled by varying the total and relative concentrations of the molecule-linker to additional linker diluent. In the most preferred embodiment, the concentration of the molecule can be adjusted in the range of micromolar to millimolar.

In order to couple the molecule to a linker, a functional coupling group is either identified on the molecule that is not involved in the biological activity to be further analyzed or attached to the molecule. Exemplary coupling groups include, but are not limited to, carboxylic acids (—COOH), primary or aliphatic amines (—RNH₂), aromatic amines or anilines (—ArNH₂), chloromethyls/vinylbenzylchlorides (—ArCH₂Cl), aldehydes (—CHO), hydrazines (NHNH₂ derivatives), hydroxyls (—OH), sulfhydryls (—SH), and epoxy groups (

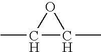

). For example, using a polypeptide molecule or an antibody, one could first identify an amine group that is not involved in binding or enzymatic activity. Useful amino acids include those containing carboxyl groups (e.g., aspartic acid and glutamic acid), hydroxyl groups (e.g., serine, threonine and tyrosine), sulfhydryl groups (e.g., cysteine) and amine groups (e.g., asparagine, glutamine, arginine, lysine and histidine). Chemical coupling may be achieved by initiating free radical species in the presence of an initiator such as peroxide, or by simply combining spontaneously chemically reactive moieties.

A variety of chemical cross-lining reagents are known to one skilled in the art. For example, primary amine reactive groups may be coupled with amine-reactive cross-linkers, such as imidoesters and N-hydroxysuccinimidyl (NHS) esters; sulfhydryl reactive groups may be coupled with sulfhydryl-reactive cross-linkers, such as maleimides, alkyl and aryl halides, α-haloacyls and pyridyl disulfides; carboxyl reactive groups can be coupled to primary amines or hydrazides using carbodiimides; and various reactive groups may be coupled with non-selective cross linkers, such as BASED (Bis-(4-azidosalicylamido)ethyl) disulfide.

Photo-activated coupling can be achieved by derivatization of the molecule with a photo-reactive moiety, such as allyl amine, arylazide, and the like, and then activating this moiety, for example with light. Ultraviolet light in the range of 320-500 nm is preferable. Photo-activation, photo-polymerization, photo-crosslinking or photo-coupling refers to a process that is activated by light. A photo-activated step can be used according to the methods of the present invention to couple a molecule to a linker and to immobilize the hydrogel to a surface. The photo-activation step may require the presence of a photoinitiator, examples of which include acetophenones, benzophenones, hydroxipropiophenones, thioxanthones, diphenyl ketones, benzoin and benzoin alkyl ethers, halogen substituted alkylaryl ketones, or quinone and anthraquinone derivatives.

Polynucleotides could be coupled to a linker via the backbone structure (i.e., any moiety other than the base groups), for example, hydroxyl groups on the phosphate groups or ribose/deoxyribose moieties. Alternatively, component bases or the 5′ or 3′ termini could provide the coupling group.

The linker contains a coupling group that will bind to the functional group identified above. For example, in order to couple a molecule containing a carboxyl group, the linker could contain an amine group; to couple a molecule containing an amine group, the linker could contain a carboxyl group; and to couple a molecule containing a sulfhydryl group, the linker could contain another sulfhydryl group. This coupling group can be attached to existing reactive groups on the hydrogel units, for example, the end hydroxyl groups of PEG polymer chains.

One of skill in the art could identify additional appropriate chemical groups, depending on the molecules to be cross-linked. Such groups could be similarly derivatized with a photo-activatable functional group.

One advantage of using a coupling group on the linker is that it may provide a “generic” coupling means for several types of molecules. By creating a few derivatives of the linker, the invention provides means of coupling several different types of the desired molecules to the hydrogel structure. In addition, the linkers for various coupling groups could readily be mixed to immobilize a library of compounds that would contain a variety of coupling groups.

Immobilization of a Hydrogel to a Surface

The hydrogel of the present invention can be immobilized to a variety of surfaces including, but not limited to, glass, metals, plastics, nylons, paper, synthetic membranes, cloth and ceramics. Preferred surfaces include single crystal silicon, polysilicon, silicon nitride, silicon dioxide, phosphosilicate glass, borophospohosilicate glass, aluminum nitride, zinc oxide, polycinylidene fluoride, lead sirconate, metal, aluminum oxide, tin oxide, tantalum oxide, and the like. Preferably, this immobilization is permanent, although it may be reversible. Exemplary steps of this immobilization comprise: contacting one or more linkers comprising the hydrogel, which preferably contains the molecule of interest, to the surface and causing a physical or chemical reaction to occur between the hydrogel and the surface via one or more linkers, preferably using chemical or photo-activated cross-linking means.

For chemical attachment of the hydrogel to the surface, presently preferred surfaces include, but are not limited to, compositions containing oxides of silicon or tungsten. In addition, a planar surface can also be silanized with an organo-silane compound, whereby a Si is coupled to the surface hydroxyl groups, to create additional reactive groups for chemical coupling. Typical silanizing agents include trimethyl cholororsilane, trichloro(3,3,3-trifluoropropyl)silane, dimethyldichlorosilane, (3-aminopropyl)-triethoxysilane, 3-acryloxyproplyltrichlorosilane, 3-methacryloxyproplyltrimethoxysilane, allyltriethoxysilane, 2-cyanoethyltriethoxysilane, 3-iodoproplytriethoxysilane, 3-bromopropyltrimethylodysilane, 3-isocyanatopropyltriethoxysilane, 3-mercaptopropyltriethoxysilane, 3-glycidoxyproplytrimethoxysilane, and similar silane derivatives.

In order to optimize the surface for the immobilization of the hydrogel, the surface can include an intermediate layer, for example, an organo-silane containing a terminal allylic, acrylic group, or a photo-polymerizable group can be used to provide additional functional groups at the surface. The hydroxyl groups existing on the surface can then bond to the Si atom, and create additional reactive species for immobilizing the hydrogel. The reaction mixture containing the molecule cross-linked with the linker is then deposited on the silanized surface. Alternatively, an intermediate layer comprising functional groups, e.g. latex, polyethylene, clock co-polymers, etc., can be placed on the surface by dip-coating, spray coating, solvent casting, spin-coating or other methods known to the skilled artisan.

Alternatively, this intermediate layer may comprise an aldehyde moiety coupled, preferably covalently, to the surface of interest. A linker derivative, for example, a hydrazine derivatized linker can then be chemically cross-linked to the aldehyde moiety of this intermediate layer.

The hydrogel is immobilized by allowing interaction between the hydrogel and the surface under conditions where a stable covalent or non-covalent linkage forms, e.g., by photo-crosslinking the hydrogel if it and the surface comprise photo-activatable groups. “Stable” in this context refers to a linkage that is not disrupted during use of the hydrogel in a subsequent procedure, e.g., under washing or binding conditions. After immobilization, the surface can then be soaked, for example, in an aqueous buffer to remove non-covalently attached hydrogel and excess cross-linking components and/or reagents.

According to the methods of the present invention, the immobilization of the hydrogel to the surface is preferably achieved in a microscale environment. The required thickness for the hydrogel at the surface will depend on the desired concentration for the immobilized molecule and the sensitivity of the detection/ analysis method. In a preferred embodiment the hydrogel is immobilized to the surface with a thickness of less than about 3000 Å, preferably less than about 1500 Å, more preferably less than about 750 Å, more preferably less than about 500 Å. To achieve optimal detection/analysis of the molecule, the thickness of the hydrogel comprising the molecule that is necessary for detection can be increased as the concentration of the molecule within the hydrogel decreases for example.

Preferably, one or more linkers comprising the hydrogel are contacted to the surface by depositing an aqueous solution directly onto the surface, which optionally may contain an intermediate layer to facilitate binding. The deposition can be performed in a variety of ways, for example, but not limited to, using a piezoelectric dispenser or dispensehead, a pin printer, or an inkjet printer. Alternative methods of contacting one or more linkers with the surface or intermediate layer, or of contacting the intermediate layer with the surface include, but are not limited to, spin coating, dip coating and Langmuir-Blodgett deposition.

In spin coating a liquid is applied to a surface which is then spun at a high rate of revolution, for example about 1000-6000 rpm. In dip coating the surface is dipped into liquid and allowed to drain prior to heating. Spin coating results in very uniform thin coatings.

Langmuir developed a method in which fatty acid monolayers on water surfaces were transferred to solid supports such as glass slides; Blodgett discovered that sequential monolayer transfer could be accomplished to form built-up multilayer films, hence the term “Langmuir-Blodgett films”. A Langmuir-Blodgett deposition procedure generally involves applying to the surface of a supporting liquid medium in a Langmuir-Blodgett trough an appropriate volume of a solution of surface active organic compound in a volatile organic solvent which preferably exhibits a rapid spreading on the subphase of supporting liquid medium. The solvent is allowed to evaporate and leave the surface active organic compound spread on the surface of the supporting liquid medium in which the organic compound is insoluble or only slightly soluble. Compression of the spread organic compound will produce a close-packed monolayer of molecules on the subphase. The supporting liquid medium usually is water, or an aqueous mixture of water and a water-miscible solvent such as ethanol or acetone. A supporting aqueous medium can contain other components such as metal ions, pH control agents, and the like. For a review of the technology see Thin Solid Films, Vol. 99(1983), which includes papers presented at the First International Conference C)n Langmuir-Blodgett Films, Durham, Great Britain, Sep. 20-22, 1982; Elsevier Sequoia S. A., Lausanne; and in Thin Solid Films, Vol. 132-134 (1985) which includes papers presented at the Second International Conference on Langmuir-Blodgett Films, Schenectady, N.Y.

Use of a Hydrogel Comprising a Molecule of Interest

According to the present invention, there are also provided methods and compositions of screening an immobilized molecule, or a library of molecules, of detecting a binding event between an immobilized molecule and a binding partner therefor, and of analyzing such a binding event. The hydrogel and methods of immobilizing a molecule of interest can be applied to any system used to detect analytes and binding events, such as those used in both clinical and non-clinical settings. Possible systems include, but are not limited to, affinity chemical sensing, arrayed sensors, and acoustic sensors.

Affinity chemical sensing systems attempt to detect interactions between a target analyte and an appropriate binding partner. Such systems generally rely on the production or use of a detectable signal. Affinity chemical sensing systems employ binding partners which can be discrete molecular species to which the target analyte specifically binds, or a phase, such as an organic polymer, into which the target partitions. Covalently attached labels such as, fluorescent, electrochemical, radioactive, or mass based-probes are typically employed in such systems. Methods for determining the presence analytes by using systems that detect the inherent optical, electrochemical, or physical properties of a target species or changes in the properties of the layer containing the binding partner to which a target species binds, have been employed to detect and/or monitor unlabeled analytes. Routinely used affinity assays are well known to those of skill in the art.

Preferably, such systems are capable of detecting interactions in a microscale environment. For example, Charych et al., U.S. Pat. No. 6,022,748, which is hereby incorporated by reference, including all tables, figures and claims, describe an example of a sensor employing an optically active sensor coating that changes color upon binding of the target. A further example of affinity sensing methods is described by W. Lukosz, Biosensors & Bioelectronics 6:215-225, 1991. Utilization of surface plasmon resonance in sensing applications is also described by Hanning in U.S. Pat. No. 5,641,640, which is hereby incorporated by reference, including all tables, figures and claims. A Chemically Selective Field Effect Transistor (CHEMFET) that determines target binding by monitoring a signal change on the sensor surface in response to target binding to the said surface, is described by Shimada in U.S. Pat. No. 4,218,298, which is hereby incorporated by reference, including all tables, figures and claims. Ribi et al., in U.S. Pat. Nos. 5,427,915 and 5,491,097, each of which is hereby incorporated by reference, including all tables, figures and claims, describe affinity-based microfabricated sensors in which a measurable change in conductivity of a bio-electric sensor layer is used to determine binding of a target species.

In preferred embodiments, the methods described herein may be used to produce arrayed sensors having multiple individually addressable sites on the device surface which are modified to contain binding partners for a target molecule to be detected. An example of such a detection system can be found in U.S. Pat. No. 6,197,503, which is hereby incorporated by reference, including all tables, figures and claims, which describes a device employing multiple optical sensing elements and microelectronics on a single integrated chip combined with one or more nucleic acid-based bioreceptors designed to detect optically labeled, sequence specific genetic constituents in complex samples.

Other examples of arrayed sensors include: U.S. Pat. No. 6,146,593, which is hereby incorporated by reference, including all tables, figures and claims, which describes a method for fabricating biosensors using functionalized optical fibers to create a high density array of uniquely addressable biological binding partners; and U.S. Pat. No. 6,124,102, which is hereby incorporated by reference, including all tables, figures and claims, which describes an optical sensor array having a planar surface derivatized with ligands of an optically active target species immobilized at known locations such that each location comprises a “pixel” of an optical read out device. These and similar devices can be successful for arrayed detection and therefore useful for parallel screening of multiple interactions where the analyte is either labeled or inherently optically, electrically, or specifically chemically active.

Another field of technology having combined arrayed sensors is that of sensors based on bulk or microfabricated resonant devices. Such sensors have been demonstrated in systems used to determine 3-dimensional acceleration, speed, and position, as transducers for monitoring environmental conditions such as pressure, fluid flow, temperature, and as gravimetrically sensitive elements in chemical affinity sensors.

Acoustic sensors for chemical sensing have been demonstrated in low-density arrays, for example, U.S. Pat. No. 4,596,697, which is hereby incorporated by reference, including all tables, figures and claims, which describes surface acoustic wave (SAW) devices. Arrays of cantilever sensors for gas phase sensing of multiple analytes are described by Lang et al., IBM Research Report, RZ 3068 (#93114), 1998, and Britton et al., Ultramicroscopy 82:17-21, 2000.

An Exemplary Microfabricated Mechanical Resonant Sensor Device

In a preferred embodiment, the methods and compositions described herein can be used to fabricate and or utilize a microfabricated mechanical resonant sensor device. For example, a molecule, or a library of molecules used for detecting or analyzing a binding event, can be performed on a surface of such a resonant sensor device comprising a hydrogel as described herein. The surface that supports the hydrogel is preferably a micromechanical sensor that detects a change in force at a membrane surface or a change in the surface properties of the sensor membrane.

The apparatus or sensor can comprise a substrate and one or more layers formed on or in the substrate. The substrate and/or layers may form a cavity comprising one or more side walls and a membrane that covers the top of the cavity. The cavity side walls can be flat, angled, sloped or curved. Preferably, the membrane provides a substantial barrier to liquid entry through the top of the cavity. The cavity also comprises at least two electrodes, which include an upper electrode and a lower electrode. The upper electrode can be the membrane itself or the upper electrode can be fabricated on, within or below the membrane. The lower electrode is below the membrane. The composition and dimension of the membrane are chosen so that it can vibrate or resonate in response to changes in electrical signal from the lower and/or upper electrode.

Preferably, the membrane is responsive to a change in resonant frequency and/or a harmonically varied electrical current. More preferably, the membrane is harmonically responsive to a change in force on the membrane surface or surface properties of the membrane, for example a binding event near and/or on the membrane surface. Preferably the diameter or width of a sensor is between at least 5 and up to 200 microns. More preferably the diameter or width of a sensor is between 10 to 100 microns.

Sensor layers that are added to the substrate during sensor fabrication can comprise single crystal silicon, polysilicon, silicon nitride, silicon dioxide, phosphosilicate glass, borophosphosilicate glass, aluminum nitride, zinc oxide, polyvinylidene fluoride, lead zirconate, metal, aluminum oxide, tin oxide, tantalum oxide, and the like. Combinations of these materials also can be used. The layers can have different electrical properties from the substrate. For example, some layers of a sensor can be used to aid in electrically isolating one region, the upper region for example, of a sensor from a lower region, or in another example, can provide electrical isolation of the membrane and the electrode(s). Layers also may be used to form electrodes and/or electrode leads. For example, a sensor layer having a cavity, can have a via, or a channel etched in the most planar surface, the XY horizontal surface of the substrate, connecting an electrode to a side wall, or the base of the cavity. This channel can be lined with a passivating layer and filled with a doped polysilicon, or a metal, such as titanium, metal alloy, titanium, gold, platinum, tungsten, aluminum, and the like, and then an additional layer having a different resistance can be placed on top of the now filled channel. In forming such layers, and in preparing electrodes and leads it would be recognized that the area of the sensor that carries an electrical charge from an electrical power source should be electrically isolated from other regions of the sensor in order to avoid a failure of conductivity, or a short, of the electrodes and leads.

The sensor layers and electrodes can be arranged by taking a substrate and applying a passivating layer, for example oxide or nitride, to provide an insulating layer between the substrate and any electrodes. Electrodes can be formed by depositing and patterning metal on the surface of the passivating layer. Another layer, a patterned spacer layer can be added with an area of the spacer layer being defined as the sensor cavity. The cavity of the sensor can be formed by etching away the defined area in the spacer layer and preferably the cavity is formed above the electrode. A membrane layer is then placed over and sealed on top of the cavity.

As used herein, “electrically isolate”, “electrical isolation”, and like terms when used in reference to electrodes, leads, arrays and sensors, refer to arranging sensors and components of sensors in a manner that insulates the array, sensor, electrode or lead that transports or carries a current of electricity from surrounding layers, sensors, electrodes or leads. For example, an array having more than one sensor in close proximity to other sensors that are electrically isolated can have essentially all of a current applied to at least one sensor in the array. Electrical isolation of sensor elements in an array can be accomplished by forming a p/n-junction between the conducting paths and the substrate. A p-n junction can be formed by doping the two halves of a single piece of a semiconductor, or two opposing layers, so that they become, respectively, p-type and n-type material, by doing this an interface is formed between the two halves creating a p-n junction. Such p-n junctions have the property that it does not allow current will flow and the junction is said to be backward biased. The sensor, array, lead or electrode can be insulated by ensuring that materials used to surround the component are incapable of conducting an electrical current. Electrical isolation could also be obtained by physically separating sensors in an array, by arranging the sensors in a manner in which they do not substantially contact another sensor yet are present on the same array.

The membrane of the sensor can be polygonal or elliptical. Preferably, it is rectangular or square having sides of 5 to 100 microns in length. More preferably, the membrane is circular having a radius of 2 and up to 100 microns, 2 to 30 or 2.5 to 50 microns in length. The membrane covers a prepared, micromachined, microfabricated or naturally occurring cavity in the substrate and can cover the cavity in a manner that prevents a liquid from entering the cavity. Preferably the membrane is up to 0.5 microns thick. More preferably the membrane is at least 0.05 and up to 0.5 microns thick.

The membrane or membrane layer of the sensor can be fabricated from an electrically conductive material, such as doped single crystal silicon, doped polysilicon, metal or any composite thereof, and can serve as a connection to ground. The membrane can be fabricated out of non-conductive materials such as silicon nitride, silicon dioxide, phosphosilicate glass, and borophosphosilicate glass. In this case, the membrane is not an electrode but can have an electrode fabricated within, on, above or below the surface. As discussed herein, the membrane covers roughly the entire opening of the cavity in a substantially sealed manner. The membrane of the sensor can also serve to conduct an electrical signal. The membrane layer can be fabricated to contain one or more secondary structures that can conduct a current of electricity such as piezoelectric or piezoresistive materials. In selecting a material to serve as a membrane for the sensor, certain mechanical characteristics such as Young's Modulus, which refers to the stiffness of the membrane, the density, the intrinsic stress, and internal damping are considered. Preferably, the membrane is prepared or fabricated in a manner that allows the membrane to vibrate and/or resonate. The membrane can also be fabricated to either serve as an electrode for conducting electricity, or as a connection to ground. The membrane can serve as part of a capacitive or electrostatic pair. As such, the membrane and the other electrode of the pair are separated by the space of the cavity and/or materials within the cavity, and act as a capacitor like structure.

The sensor may comprise a cavity that is preferably 0.1 to 2 microns deep and sealed with an addressable, conductive membrane. Preferably, the membrane is preferably 0.1 to 0.5 microns in thickness. The sensor membrane can resonate or vibrate and can be used as a chemical affinity sensor. For example, the topmost surface of the membrane can be derivatized to support the immobilized hydrogel comprising at least one member of a binding pair. Upon exposure to a liquid or gas phase that contains a second member of the binding pair, binding occurs between the two molecules and an increase in mass relative to the mass of the single membrane bound member occurs on the membrane. This change in mass at the surface of the membrane can alter the resonant characteristics of the membrane and/or the fundamental frequency of vibration or the phase of a vibration relative to a driving signal of the membrane can be said to change and/or shift.

By the term “addressable” when describing the electrical potential of the sensor, membrane, electrode or an array, is meant that the described layer, sensor, substrate and/or membrane can accept an electron, have an electric potential or voltage assignment. The electric potential can be the assignment of having a ground voltage, such as for example the membrane can be held at ground voltage when a sensor operates using an AC, alternating current, power source, or the assignment can be a lower or higher electric potential within the membrane in reference to an opposing electrode if using a DC, direct current, electrode power source. The term “addressable” when used to describe a sensor, whether in a single array or in separate arrays, combines the concept of assigning an electric potential or voltage and relates to each sensor being capable of being given a specific locator and/or identifier, allowing a particular sensor in an array to be separately identifiable from surrounding sensors when used in methods such as high through put screening.

As discussed above, the sensor comprises a cavity that is covered by a membrane as described herein. Preferably, the cavity comprises one or more walls that are 0.1 to 2 microns in height. More preferably, the cavity walls are 0.3 to 1 micron in height, creating a cavity or a well that is roughly 0.3 to 1 micron deep. It is the distance between the upper electrode and lower electrode that determines the height of the cavity. Thus, the distance from the lower to upper electrode is roughly 0.3 to 1. Preferably, the cavity is completely covered in a sealed, liquid free manner by the membrane. The membrane that completely covers the cavity may comprise one or more holes on its surface.

The cavity may serve as the dielectric space between the electrode in the bottom of the cavity and the upper electrode. It would be understood by one of ordinary skill in the art that combination of the lower and upper electrode comprises a capacitor system where the cavity is, as stated, the dielectric space through which an electrostatic field can be formed.

The cavity of the sensor may contain passages, or vents, which are holes in the substrate that connect the exterior of the sensor to the cavity. Preferably, the cavity can be vented, having holes, tunnels or pores that allow the cavity to be vented to the external atmosphere of the sensor. Vents in the cavity of the sensor function to eliminate and/or relieve the effects of barometric pressure variation and pressurization in the cavity during operation of the device. The passages or vents can be in the base, the sidewalls of the cavity, or in the membrane which covers the cavity. Thus, in this configuration, the sensor has a sealed cavity being covered by a membrane having vents. The cavity can be partially filled, or filled with a dielectric or multiple dielectric materials or gases, such as tantalum, polypropylene film, polymer-aluminum, polyester, metalized polyester, plastic foam sheet, transformer oils such as paraffin, gasses such as argon, oxygen, chlorine, and any mixture of the like. Preferably, the cavity comprises at least one passage or vent that passes in the Z or perpendicular dimension, through the substrate and to the exterior and/or ambient surroundings of the sensor. The vents and/or passages can extend in a horizontal or planar manner from the cavity through the walls of the cavity well or the vents and/or passages can extend through the floor of the cavity leading in a perpendicular manner to the exterior of the sensor.

The sensor can comprise at least two electrodes as mentioned above. Electrodes in the sensor cavity are preferably planar. The electrodes of the sensor can be created by etching vias or channels into the substrate, providing a passivating insulating layer and implanting or sputtering a metal, for example titanium, gold, platinum, tungsten, a metal alloy and/or other like metals. One may also dope the substrate with an impurity which depending on the type of substrate chosen, p-type or n-type, can indicate either a substance such as boron, P-type, or phosphorus, N-type, to act as leads and/or electrodes. The electrodes of the sensor can from one or more metal or diffused dopant electrode layers in the bottom of the cavity.

Leads are used to connect electrodes to a power source or ground. The leads can be prepared by fabricating holes or tunnels through the substrate cavity, which lead away from the cavity in a perpendicular manner. Such perpendicular leads can be prepared to extend through the substrate to the exterior of the sensor in order to be connected to an electrical current source, or the leads can extend from the substrate cavity floor and be configured to exit the sensor at an angle, through one or more sides of the sensor itself.

The electrodes can be part of, or reside on or within the membrane of the sensor. One of ordinary skill in the art would recognize that preparation of electrodes on, within or under the membrane should not interfere with either the acoustics of the cavity, nor should they interfere with the resonance, oscillation or vibrations of the membrane itself. One or more electrodes may be prepared on, within or under the membrane to serve as actuating electrodes and/or sensing electrodes, and it is also possible to have electrodes on, within or under the membrane and have additional electrodes as described above.

Resonation or vibration of the membrane can be initiated electrostatically through use of electrodes in the sensor base, the membrane, the cavity wall, the cavity floor and/or membrane where the electrodes are connected in a manner that allows the initiation or creation of an electric current and/or potential. Resonation or vibration of the membrane of the sensor can be monitored using electrodes that can be located in and around the sensor as described and illustrated herein, and which can be part of a monitor apparatus, or monitoring can occur, for example, either acoustically, electronically by electromechanical transduction such as by electrostatics/ capacitance, piezoresistance or piezoelectricity, or optically by interferometry, such as laser-Doppler vibrometery.

The above described sensor(s) can be arranged in an array from as few as a handful of sensor sites to as many as 500,000 individual sensors per cm². High-density arrays can comprises between 256 to 150,000 individual sensors/ cm², and more preferably between 5,000 to 100,000 sensors/cm². Each sensor in the array can be fabricated to generally function similarly. However, individual sensor sites may have different types of sensors, which differ in their mode of operation. It is preferred that individual sensor sites are arranged in the array in a manner that allows for electrical isolation of each sensor. The individual sensor sites may be individually addressed. Multiple sensor sites may be linked so that they can be actuated and detected simultaneously.

High-density arrays having multiple sensors where individual sensors of the array have a cavity differing in width, depth and shape are also contemplated. The individual sensors of an array can also comprise membranes of different width and composition. For example, one or more sensors of an array can comprise membranes with or without holes on their surfaces. Preferably, the individual sensors of high-density arrays comprise the same cavity shape and depth and further comprise membranes and substrates of the same width and composition. More preferably, the individual sensors of an array are essentially identical in shape and composition, and are individually addressable. A high-density array can also comprise sensors that are grouped together to detect the same analyte.

Arrays can be used in multiplexed assays, which can be considered assays where more than one analyte is detected in a sample. For example, an array can be prepared with multiple sensors each having different binding partners. A sample believed to contain any of the multiple analytes of interest can be placed in contact with the sensor array and various individual sensors can be monitored, based on the membrane response, to determine which analytes are present in the sample. It would be understood by those of skill in the art that more than one sensor site in the array can be used as a control or reference sensor to determine reference values for the assay, such as the baseline response for the membranes. It would also be recognized by those skilled in the art that due to the size of the arrays, multiple arrays with probes for multiple targets can be used simultaneously.

Exemplary Types of Screening or Binding Assays

In further aspects of the present invention, there are provided methods of screening an immobilized molecule, or a library thereof, preferably using such a microfabricated resonant sensor device. A hydrogel comprising said molecule, or library thereof is immobilized to a surface and contacted with a sample suspected of containing or known to contain a binding partner for said molecule. A binding reaction between the molecule and a binding partner within the sample is then detected. Numerous methods to detect signals from binding assays are known to the skilled artisan. In preferred embodiments! the binding reaction may result in a change in the surface, typically a result of a change in mass, which can be detected, e.g., by a resonant sensor device.

A binding reaction may be detected directly, or alternatively, following amplification of the binding event. For example, when the amount of analyte that binds to a sensor surface is too low for the sensor to detect, the sensor can be contacted with a sample containing a second binding partner specific for the analyte bound to the sensor membrane. The sample-containing binding partner is preferably specific for a site on the analyte that is separate and non overlapping from the site bound by the membrane immobilized binding partner, so that the two binding partners can bind simultaneously to a single analyte molecule. Indirect detection can be achieved when the additional mass attributed to binding of the second binding partner to analyte on the membrane becomes detectable. Alternatively, the second binding partner may contain an enzyme able to catalyze deposition of additional material in an analyte dependent manner, see, e.g., U.S. Pat. No. 6,280,961, which is hereby incorporated by reference, including all tables, figures and claims.

Competitive inhibition may be used with a sensor or sensor array of the invention when an inhibitor analyte of lower mass inhibits binding of a larger mass analyte to the membrane.

In one aspect of the invention, detecting the presence or amount of one or more analyte(s) in a sample can be used in screening procedures as described herein. Exemplary steps of this screening method comprise: acquiring a sample presumed to contain or known to contain one or more analyte(s) to be detected, contacting the sample with a hydrogel comprising binding partners for each analyte, and determining the presence of the analyte(s) in the sample. In a preferred embodiment this detection step is based on a measured detectable change in membrane resonance or vibration. In various embodiments, the analyte(s) to be detected and/or the binding partner(s) of the analyte(s) are not labeled and the sample is a liquid or gas.

In another aspect, the rate of binding of a known amount of one or more analyte(s) in a sample to one or more binding partners immobilized in a hydrogel can be analyzed. Exemplary steps of this screening method comprise: contacting a hydrogel comprising the binding partner(s) with the sample and detecting a change in the binding event over a period of time. In preferred embodiments, the rate of binding that occurs over time correlates to the rate constant of reaction between an analyte and its binding partner.

In another aspect, the affinity between one or more analyte(s) and the corresponding binding partner(s) can be analyzed. Exemplary steps of this screening method comprise: contacting a sample containing a known concentration of the analyte(s) with a hydrogel comprising the binding partner(s) with the sample; detecting change in the binding event over a period of time; removing the first sample and repeating these contacting and detecting steps with a different concentration of the analyte(s). This cycle of removal, contacting with a different analyte concentration and measuring the binding event over time may be repeated multiple times, each with a different analyte concentration. The binding affinity between an analyte and its binding partner can be derived by relating binding rate to analyte concentration in a manner well known to those of skill in the art.

The array of sensors may be designed to operate with a parallel array of molecular probes. Each site within the array can be derivatized with a different molecule such that the device becomes potentially chemically responsive to sample solutions. In a preferred embodiment, a binding event between a substance in a sample solution and a molecule within the hydrogel results in an increase of the surface mass of the membrane and a corresponding decrease in resonant frequency or vibration. Screening is designed to be performed under wet conditions and does not necessitate drying of the sensor. Doing so could alter the chemical reactivity of the involved species, cause denaturing, conformational changes, or instabilities in the substances, and create problems such as the precipitation of salt from solution. For further application details, refer to U.S. Pat. No. 5,912,181, which is hereby incorporated by reference, including all tables, figures and claims. Chemical binding constants and affinity can be determined as described above by titration of the sample solution over the device and real-time monitoring of resonant frequency shifts as a function of concentration. The preferred sensor device is also robust enough to be reusable such that multiple samples can be serially flowed over sensor and screened in sequence.

The array screening methods and compositions described herein may be applied to pharmaceutical high throughput screening. For example, in a preferred embodiment, the activity of a molecule such as a receptor or enzyme against an entire combinatorial library can be performed in parallel. Each member of the library would be immobilized to an individual membrane. A solution containing the molecule is passed over the entire sensor and “hits” are identified by locating the sites that displayed mass-induced resonant frequency shifts. Multiple screenings of various molecules against the same library can be performed on a single derivatized sensor by sequentially flowing various test solutions containing the desired molecules and wash solutions over the chip. Binding constants of hits can also be measured by titration of samples.

The invention will now be described in greater detail by reference to the following non-limiting examples.

### Example 1

### Materials and Reagents

Poly (ethylene glycol) bisphenol a-glycidyl ether tetra acrylate (PEG-TA) (MW 18,500), 3-(trimethoxy silyl) propyl methaacrylate (TPM) were obtained from Polysciences. 2,2′ dimethoxy-2-phenylacetophenone (DMPA), anhydrous, carbon tetrachloride, anhydrous toluene, n-heptane, 1-vinyl-2-pyrrolidone, diisopropylethylamine (DIEA), sulfuric acid, allylamine, dicyclohexylamine (DCC) were purchased from Aldrich (St. Paul, Minn.). Streptavidin, biotinylated goat IgG, anti-goat IgG, and succinimidyl-6-(biotinamido) heaxanoate (NHS-LC-Biotin) were purchased from Pierce (Rockford, Ill.). 30% Hydrogen peroxide was purchased from J. T. Baker.

### Example 2

### Derivatization of Amino Acids to Incorporate a Photoactive Functional Group

The photoactive group is provided by an allyl amino, which was used to synthesize allylamido-6-(biotinamido) hexanoate, producing a biotin molecule with a photoreactive functional group. NHS-LC-Biotin (MW 454, 1 mmole) was reacted with allyl amine (MW 57, 1 mmole), DCC (MW 206,2 mmole) and DIEA (5%) in 50 mL DMF for 24 hr. The product was analyzed by TLC and purified by HPLC.

### Example 3—Deposition of a PEG Hydrogel Comprising a Molecule on a Surface

The surface was rinsed with deionized water, and dried at 150° C. for 30 minutes under nitrogen. The surface was then treated for 5 minutes in an N₂ atmosphere at room temperature with a solution of 1 mM of TPM and 1 mM DIEA in a 4:1 mixture of heptane:CCl. The surface was subsequently rinsed with hexane and dried under N₂ at 150° C. for 30 minutes. Such surface modifications are described in Revzin et al., Langmuir 17:5440-5447, 2001; and Vandenberg et al., J. Coll. Interf. Soc. 147:103, 1991.

PEG molecules were then photo-linked to the surface and to the biotin molecule. A macromer solution was prepared by dissolving 150 mg of PEG-TA in 1 ml of warm 1:1 H₂0: EtOH. A photo-activatable solution was prepared by adding 10 μL of a photoinitiator solution (600 mg of DMPA in 1 ml of 1-vinyl-2-pyrrolidinone), and 10 μL of the allyl modified biotin (2.8 mg in 1 ml of ETOH) to the macromer solution. An aliquot of the photo-activatable solution was deposited on the surface and the surface was spun at 2000 rpm for 30 seconds to form a thin film coating. This coating was irradiated with UV light (filtered to pass 320-500 nm, EFOS Novacure) for 30 seconds at 4000 mW. The surface was then coated with a PEG hydrogel comprising the biotin molecule, wherein the PEG was covalently coupled to the biotin, and the PEG was covalently coupled to the surface. The hydrogel-coated surface was then rinsed in deionized water, ultrasonically cleaned in water for 10 seconds to remove unpolymerized gel, and rinsed again in deionized water. The hydrogel comprising the biotin molecule immobilized to the surface can be stored at 4° C. in a humid atmosphere to prevent drying from occurring.

### Example 4

### Binding Assay with an Hydrogel Immobilized Molecule on a Resonant Sensor

A sensor can be made as described in the Patent Application filed with the USPTO on Sept. 20, 2001, entitled “Microfabricated Ultrasound Array for Use as Resonant Sensors” (U.S. application Ser. No. 09/957,875), the contents of which are hereby incorporated by reference in their entirety, including all tables, figures, references and claims.

In order to indirectly couple a binding partner molecule to the sensor, a 5 micromolar solution of streptavidin in water is contacted with the hydrogel. For a sensor with a surface area of approximately 1 cm², the sensor is submerged in about 300 μl of the streptavidin solution. The surface is then rinsed with a 0.1 M phosphate buffer (pH 6). This results in attachment of streptavidin within the hydrogel. Exposure of the surface to a 1 micromolar solution of a biotinylated antigen, such as goat IgG, in the same buffer immobilizes the antigen within the hydrogel at the sensor surface.

For determination of goat IgG-anti-goat IgG interaction parameters, the derivatized sensor containing the immobilized molecule within the hydrogel is placed within a flow cell in contact with a buffer solution and the resonant frequency of the sensor is measured. Subsequently, a solution of the same buffer containing a known concentration anti-goat IgG is introduced into the flow cell and the change of the resonant frequency is monitored. Upon achieving equilibrium, the solution in the flow cell is changed to one containing a second known concentration of antibody. Again the rate and magnitude of the change in the resonant frequency is determined. Multiple solutions of various known concentrations of antibody may be used. The results from these measurements are used to determine the kinetic and equilibrium constants for the antibody-antigen interaction.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

That which is claimed is:

1. A method for the microscale immobilization of a molecule at a discrete location on a surface, said method comprising:
forming a hydrogel comprising said molecule from a plurality of linkers at said discrete location, wherein one or more of said linkers bind said molecule, and one or more of said linkers bind said discrete location to form an immobilized hydrogel having a volume up to 1 microliter.

2. The method of claim 1, wherein said linkers contain a functional group on at least one end.

3. The method of claim 2, wherein said functional group is photo-activatable.

4. The method of claim 2, wherein said functional group is chemically activatable.

5. The method of claim 2, wherein said functional group comprises an acrylic group.

6. The method of claim 2, wherein said functional group comprises an allylic group.

7. The method of claim 1, wherein said molecule is covalently bound to one or more of said linkers.

8. The method of claim 1, wherein one or more of said linkers bind said molecule via photo-crosslinking.

9. The method of claim 1, wherein one or more of said linkers bind said molecule via chemical crosslinking.

10. The method of claim 1, wherein one or more of said linkers is covalently bound to said surface.

11. The method of claim 1, wherein one or more of said linkers bind said surface via photo-crosslinking.

12. The method of claim 1, wherein one or more of said linkers bind said surface via chemical crosslinking.

13. The method of claim 1, wherein said surface comprises a microfabricated resonant sensor.

14. The method of claim 1, wherein said forming step comprises contacting said discrete location with said plurality of linkers and binding one or more of said linkers to said surface.

15. The method of claim 1, wherein said forming step comprises polymerizing said plurality of linkers to form a hydrogel contacting said hydrogel to said discrete location, and binding said hydrogel to said surface.

16. The method of claim 1, wherein said hydrogel is formed at a plurality of discrete locations on said surface.

17. The method of claim 1, wherein said hydrogel is formed by covalently binding said molecule to one or more of said linkers, wherein said linkers contain a photo-activatable functional group, and wherein one or more of said linkers is covalently bound to said surface by photo-induced activation of said functional group.

18. The method of claim 1, wherein said immobilized hydrogel has a volume of 0.1 picoliters to 1 microliter.

19. The method of claim 1, wherein said molecule is a polypeptide or peptide.

20. The method of claim 1, wherein said molecule is a chemical compound.

21. The method of claim 1, wherein said molecule is an antibody.

22. The method of claim 1, wherein said molecule is a polynucleotide.

23. The method of claim 1, wherein said molecule is a carbohydrate.

24. The method of claim 1, wherein said linkers are polyethylene glycol, branched polyethylene glycol, star polyethylene glycol, a block co-polymer of polyethylene glycol or a derivative of polyethylene glycol.

25. The method of claim 1, wherein said linkers are polypropylene glycol, branched polypropylene glycol, star polypropylene glycol, a block co-polymer of polypropylene glycol or a derivative of polypropylene glycol.

26. The method of claim 1, wherein said linkers are polylysine, or a block co-polymer or derivative thereof.

27. A method of screening an immobilized molecule, wherein a hydrogel comprising said molecule is formed from a plurality of linkers, wherein one or more of said linkers bind said molecule, and one or more of said linkers bind a surface, said method comprising:
contacting said hydrogel with a sample suspected of containing or known to contain a binding partner for said molecule; and
detecting a binding event between said molecule and a binding partner for said molecule.

28. The method of claim 27, wherein the binding event is detected through a chemically-generated color change, fluorescence, or luminescence.

29. The method of claim 27, wherein said surface comprises a microfabricated resonant sensor.

30. The method of claim 29, wherein the binding event is detected by monitoring the resonant frequency of the sensor.

31. The method of claim 27, wherein said molecule is selected from the group consisting of a polypeptide, a peptide, a chemical compound, an antibody, a polynucleotide and a carbohydrate.

32. The method of claim 27, wherein said hydrogel comprises a library of said molecules.

33. A method of detecting a binding event between an immobilized molecule and a binding partner for said molecule, wherein a hydrogel comprising said molecule is formed from a plurality of linkers, wherein one or more of said linkers bind said molecule, and one or more of said linkers bind a surface, said method comprising:
contacting said hydrogel with a sample suspected of containing or known to contain a binding partner for said molecule; and
detecting said binding event.

34. The method of claim 33, wherein the binding event is detected through a chemically-generated color change, fluorescence, or luminescence.

35. The method of claim 33, wherein said surface comprises a microfabricated resonant sensor.

36. The method of claim 35, wherein the binding event is detected by monitoring the resonant frequency of the sensor.

37. The method of claim 33, wherein said molecule is selected from the group consisting of a polypeptide, a peptide, a chemical compound, an antibody, a polynucleotide and a carbohydrate.

38. The method of claim 33, wherein said hydrogel comprises a library of said molecules.

39. A method of analyzing a binding event between an immobilized molecule and a binding partner for said molecule, wherein a hydrogel comprising said molecule is formed from a plurality of linkers, wherein one or more of said linkers bind said molecule, and one or more of said linkers bind a surface, said method comprising:
contacting said hydrogel with a sample suspected of containing or known to contain a binding partner for said molecule; and
analyzing said binding event.

40. The method of claim 39, wherein the binding event is detected through a chemically-generated color change, fluorescence, or luminescence.

41. The method of claim 39, wherein said surface comprises a microfabricated resonant sensor.

42. The method of claim 41, wherein the binding event is detected by monitoring the resonant frequency of the sensor.

43. The method of claim 39, wherein said molecule is selected from the group consisting of a polypeptide, a peptide, a chemical compound, an antibody, a polynucleotide and a carbohydrate.

44. The method of claim 39, wherein said hydrogel comprises a library of said molecules.

45. A hydrogel for the microscale incorporation of molecules, formed by associating a plurality of linkers, wherein one or more of said linkers bind said molecule, and one or more of said linkers bind a surface.

46. The hydrogel of claim 45, wherein said linkers contain a functional group on at least one end.

47. The hydrogel of claim 46, wherein said functional group is photo-activatable.

48. The hydrogel of claim 46, wherein said functional group is chemically activatable.

49. The hydrogel of claim 46, wherein said functional group comprises an acrylic group.

50. The hydrogel of claim 46, wherein said functional group comprises an allylic group.

51. The hydrogel of claim 45, wherein one or more of said linkers is covalently bound to said surface.

52. The hydrogel of claim 45, wherein said surface comprises a microfabricated resonant sensor.

53. The hydrogel of claim 45, further comprising a small molecule.

54. The hydrogel of claim 53, wherein said molecule is covalently bound to one or more of said linkers.

55. The hydrogel of claim 53, wherein said molecule is selected from the group consisting of a polypeptide, a peptide, a chemical compound, an antibody, a polynucleotide and a carbohydrate.

56. A library of molecules incorporated within a hydrogel, wherein said hydrogel is formed from a plurality of linkers, wherein one or more of said linkers bind said molecule, and one or more of said linkers bind a surface.

57. The library of claim 56, wherein said molecules are selected from the group consisting of polypeptides, peptides, chemical compounds, antibodies, polynucleotides, and carbohydrates.

58. An apparatus for the detection and/or analysis of a binding event between a molecule and a binding partner for said molecule, said apparatus comprising a hydrogel comprising said molecule is formed by associating a plurality of linkers, wherein one or more of said linkers bind said molecule, and one or more of said linkers bind a surface.

59. The apparatus of claim 58, wherein said surface comprises a microfabricated resonant sensor.

60. The apparatus of claim 58, wherein the apparatus is a microfabricated resonant sensor device or a derivative or improvement thereof.
